(19) United States
(12) Patent Application Publication
(10) Pub. No.: US 2025/0149035 A1
(43) Pub. Date: May 8, 2025

(54) PERSONALIZED VIRTUAL ASSISTANCE FOR HEARING INSTRUMENT USERS

(71) Applicant: Starkey Laboratories, Inc., Eden Prairie, MN (US)

(72) Inventors: Majd Srour, San Jose, CA (US); Dean G. Meyer, Mound, MN (US); Alec Roberts Jeffery, Bloomington, MN (US); David A. Fabry, Eden Prairie, MN (US); Jon Kindred, Minneapolis, MN (US)

(21) Appl. No.: 18/933,611

(22) Filed: Oct. 31, 2024

Related U.S. Application Data
(60) Provisional application No. 63/596,487, filed on Nov. 6, 2023.

(51) Int. Cl. G10L 15/22; G10L 15/18; G16H 10/60; G16H 20/10; H04R 25/00

(52) U.S. Cl. CPC G10L 15/22; G10L 15/1815; G16H 10/60; G16H 20/10; H04R 25/00; G10L 2015/223; G10L 2015/228

(57) ABSTRACT

A method comprising: receiving, by a computing system, audio data generated by one or more hearing instruments worn at or near one or more ears of a user; providing, by the computing system, a virtual personal assistant to the user, wherein the virtual personal assistant is configured to generate, based on the audio data, output to assist the user, wherein providing the virtual personal assistant comprises applying, by the computing system, a Large Language Model (LLM) to generate a response, and the output is based on the response; and providing, by the computing system, the output to the one or more hearing instruments, wherein the one or more hearing instruments are configured to generate auditory stimuli based on the output.

This application claims priority to U.S. Provisional Patent Application 63/596,487, filed Nov. 6, 2023, the entire content of which is incorporated by reference.

### TECHNICAL FIELD

This disclosure relates to hearing instruments.

### BACKGROUND

Hearing instruments are devices designed to be worn on, in, or near one or more of a user's ears. Common types of hearing instruments include hearing assistance devices (e.g., “hearing aids”), earbuds, headphones, hearables, cochlear implants, and so on. In some examples, a hearing instrument may be implanted or integrated into a user. Some hearing instruments include additional features beyond just environmental sound-amplification. For example, some modern hearing instruments include advanced audio processing for improved functionality, controlling and programming the hearing instruments, wireless communication with external devices including other hearing instruments (e.g., for streaming media), and so on.

### SUMMARY

In general, this disclosure describes techniques related to the use of artificial intelligence to provide a virtual personal assistant for users of hearing instruments. The details of one or more aspects of the disclosure are set forth in the accompanying drawings and the description below. Other features, objects, and advantages of the techniques described in this disclosure will be apparent from the description, drawings, and claims.

In one example, this disclosure describes a method comprising: receiving, by a computing system, audio data generated by one or more hearing instruments worn at or near one or more ears of a user; providing, by the computing system, a virtual personal assistant to the user, wherein the virtual personal assistant is configured to generate, based on the audio data, output to assist the user; and providing, by the computing system, the output to the one or more hearing instruments, wherein the one or more hearing instruments are configured to generate auditory stimuli based on the output.

In another example, this disclosure describes a computing system comprising: one or more memories; and one or more processors configured to: receive audio data generated by one or more hearing instruments worn at or near one or more ears of a user; provide a virtual personal assistant to the user, wherein the virtual personal assistant is configured to generate, based on the audio data, output to assist the user, wherein as part of providing the virtual personal assistant, the one or more processors apply a Large Language Model (LLM) to generate a response, and the output is based on the response; and provide the output to the one or more hearing instruments, wherein the one or more hearing instruments are configured to generate auditory stimuli based on the output.

In another example, this disclosure describes one or more non-transitory computer-readable media having instructions stored thereon that, when executed by one or more processors of a computing system, cause the one or more processors to: receive audio data generated by one or more hearing instruments worn at or near one or more ears of a user; provide a virtual personal assistant to the user, wherein the virtual personal assistant is configured to generate, based on the audio data, output to assist the user, wherein providing the virtual personal assistant comprises applying a Large Language Model (LLM) to generate a response, and the output is based on the response; and provide the output to the one or more hearing instruments, wherein the one or more hearing instruments are configured to generate auditory stimuli based on the output.

In another example, this disclosure describes a computer-implemented method comprising: obtaining, by one or more processors of a computing system, audio data representing voice input from a user of one or more hearing instruments, wherein the voice input is detected by one or more microphones of the one or more hearing instruments; providing, by the one or more processors, a first input to a generative artificial intelligence (AI) system, wherein the first input requests that the generative AI system generate a first output that indicates whether the voice input is in a real-time information request class, wherein voice inputs in the real-time information request class request information beyond training information on which the generative AI system was trained; based on the first output indicating that the voice input is in the real-time information request class, sending, by the one or more processors, via one or more communication links external to the computing system, a request for a real-time data service to provide real-time information specified by the voice input; receiving, by the one or more processors, the real-time information from the real-time data service; providing, by the one or more processors, a second input to the generative AI system, wherein the second input requests that the generative AI system generate a second output that expresses the real-time information as a personal assistant would provide the real-time information in a conversation between the personal assistant and the user; and transmitting, by the one or more processors, to the one or more hearing instruments, response data that cause the one or more hearing instruments to output sound representing the second output.

In another example, this disclosure describes a computer-implemented method comprising: obtaining, by one or more processors of a computing system, audio data representing voice input from a user of one or more hearing instruments, wherein the voice input is detected by one or more microphones of the one or more hearing instruments; providing, by the one or more processors, a first input to a generative artificial intelligence (AI) system, wherein the first input requests that the generative AI system generate a first output that indicates whether the voice input is in a real-time information request class, wherein voice inputs in the real-time information request class request information beyond training information on which the generative AI system was trained; based on the first output indicating that the voice input is in the real-time information request class, sending, by the one or more processors, via one or more communication links external to the computing system, a request for a real-time data service to provide real-time information specified by the voice input; receiving, by the one or more processors, the real-time information from the real-time data service; providing, by the one or more processors, a second input to the generative AI system, wherein the second input requests that the generative AI system generate a second output that expresses the real-time information as a personal assistant would provide the real-time information in a conversation between the personal assistant and the user; and transmitting, by the one or more processors, to the one or more hearing instruments, response data that cause the one or more hearing instruments to output sound representing the second output.

### BRIEF DESCRIPTION OF DRAWINGS

FIG. 1 is a conceptual diagram illustrating an example system that includes one or more hearing instruments, in accordance with one or more techniques of this disclosure.

FIG. 2 is a block diagram illustrating example components of a hearing instrument, in accordance with one or more techniques of this disclosure.

FIG. 3 is a block diagram illustrating example components of a computing device, in accordance with one or more techniques of this disclosure.

FIG. 4 is a block diagram illustrating example components of a virtual personal assistant, in accordance with one or more techniques of this disclosure.

FIG. 5 is a flowchart illustrating an example operation of the virtual personal assistant, in accordance with one or more techniques described in this disclosure.

FIG. 6 is a flowchart illustrating an example operation, in accordance with one or more techniques of this disclosure.

FIG. 7 is a conceptual diagram illustrating an example software architecture, in accordance with one or more techniques of this disclosure.

### DETAILED DESCRIPTION

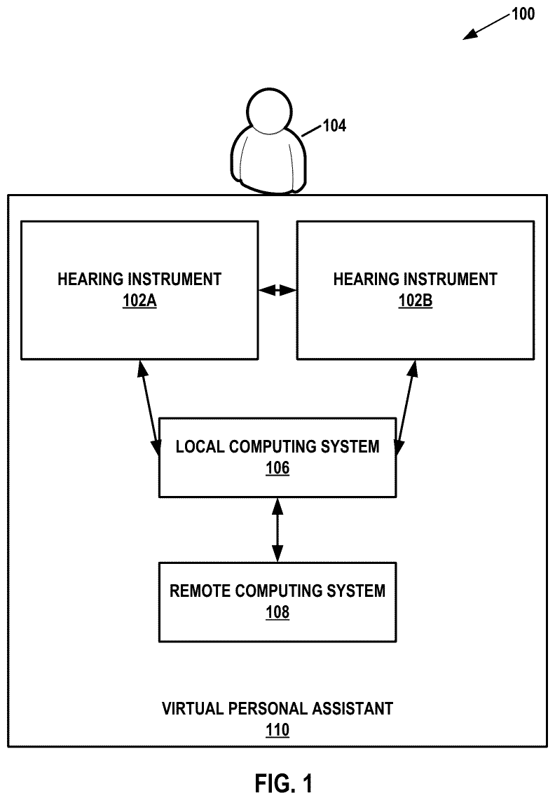

FIG. 1 is a conceptual diagram illustrating an example system 100 that includes hearing instruments 102A, 102B, in accordance with one or more techniques of this disclosure. This disclosure may refer to hearing instruments 102A and 102B collectively, as “hearing instruments 102.” A user 104 may wear hearing instruments 102. In some instances, user 104 may wear a single hearing instrument. In other instances, user 104 may wear two hearing instruments, with one hearing instrument for each ear of user 104.

Hearing instruments 102 may include one or more of various types of devices that are configured to provide auditory stimuli to user 104 and that are designed for wear and/or implantation at, on, near, or in relation to the physiological function of an ear of user 104. Hearing instruments 102 may be worn, at least partially, in the ear canal or concha. One or more of hearing instruments 102 may include behind the ear (BTE) components that are worn behind the ears of user 104. In some examples, hearing instruments 102 include devices that are at least partially implanted into or integrated with the skull of user 104. In some examples, one or more of hearing instruments 102 provides auditory stimuli to user 104 via a bone conduction pathway.

In any of the examples of this disclosure, each of hearing instruments 102 may include a hearing assistance device. Hearing assistance devices include devices that help user 104 hear sounds in the environment of user 104. Example types of hearing assistance devices may include hearing aid devices, Personal Sound Amplification Products (PSAPs), cochlear implant systems (which may include cochlear implant magnets, cochlear implant transducers, and cochlear implant processors), bone-anchored or osseointegrated hearing aids, and so on. In some examples, hearing instruments 102 are over-the-counter, direct-to-consumer, or prescription devices. Furthermore, in some examples, hearing instruments 102 include devices that provide auditory stimuli to user 104 that correspond to artificial sounds or sounds that are not naturally in the environment of user 104, such as recorded music, computer-generated sounds, or other types of sounds. For instance, hearing instruments 102 may include so-called “hearables,” earbuds, earphones, or other types of devices that are worn on or near the ears of user 104. Some types of hearing instruments provide auditory stimuli to user 104 corresponding to sounds from the user's environment and also artificial sounds. In some examples, hearing instruments 102 may include cochlear implants or brainstem implants. In some examples, hearing instruments 102 may use a bone conduction pathway to provide auditory stimulation. In some examples, one or more of hearing instruments 102 includes a housing or shell that is designed to be worn in the ear for both aesthetic and functional reasons and encloses the electronic components of the hearing instrument. Such hearing instruments may be referred to as in-the-ear (ITE), in-the-canal (ITC), completely-in-the-canal (CIC), or invisible-in-the-canal (IIC) devices. In some examples, one or more of hearing instruments 102 may be behind-the-ear (BTE) devices, which include a housing worn behind the ear that contains all of the electronic components of the hearing instrument, including the receiver (e.g., a speaker). The receiver conducts sound to an earbud inside the ear via an audio tube. In some examples, one or more of hearing instruments 102 are receiver-in-canal (RIC) hearing-assistance devices, which include housings worn behind the ears that contains electronic components and housings worn in the ear canals that contains receivers.

Hearing instruments 102 may implement a variety of features that help user 104 hear better. For example, hearing instruments 102 may amplify the intensity of incoming sound, amplify the intensity of certain frequencies of the incoming sound, translate or compress frequencies of the incoming sound, receive wireless audio transmissions from hearing assistive listening systems and hearing aid accessories (e.g., remote microphones, media streaming devices, and the like), and/or perform other functions to improve the hearing of user 104. In some examples, hearing instruments 102 implement a directional processing mode in which hearing instruments 102 selectively amplify sound originating from a particular direction (e.g., to the front of user 104) while potentially fully or partially canceling sound originating from other directions. In other words, a directional processing mode may selectively attenuate off-axis unwanted sounds. The directional processing mode may help user 104 understand conversations occurring in crowds or other noisy environments. In some examples, hearing instruments 102 use beamforming or directional processing cues to implement or augment directional processing modes.

In some examples, hearing instruments 102 reduce noise by canceling out or attenuating certain frequencies. Furthermore, in some examples, hearing instruments 102 may help user 104 enjoy audio media, such as music or sound components of visual media, by outputting sound based on audio data wirelessly transmitted to hearing instruments 102.

Hearing instruments 102 may be configured to communicate with each other. For instance, in any of the examples of this disclosure, hearing instruments 102 may communicate with each other using one or more wireless communication technologies. Example types of wireless communication technology include Near-Field Magnetic Induction (NFMI) technology, 900 MHz technology, BLUETOOTH™ technology, WI-FI™ technology, audible sound signals, ultrasonic communication technology, infrared communication technology, inductive communication technology, or other types of communication that do not rely on wires to transmit signals between devices. In some examples, hearing instruments 102 use a 2.4 GHz frequency band for wireless communication. In examples of this disclosure, hearing instruments 102 may communicate with each other via non-wireless communication links, such as via one or more cables, direct electrical contacts, and so on.

As shown in the example of FIG. 1, system 100 may also include a local computing system 106 and a remote computing system 108. In other examples, system 100 does not include one or more of local computing system 106 or remote computing system 108. Each of local computing system 106 and remote computing system 108 may include one or more computing devices, each of which may include one or more processors. In general, local computing system 106 is local to, e.g., carried by, worn by, or otherwise in the vicinity of, user 104. For instance, local computing system 106 may include one or more mobile devices (e.g., smartphones, tablet computers, etc.), handheld devices, wireless access points, smart speaker devices, smart televisions, medical alarm devices, smart key fobs, smartwatches, smart displays, screen-enhanced smart speakers, wireless routers, wireless communication hubs, prosthetic devices, mobility devices, special-purpose devices, accessory devices, and/or other types of devices. Accessory devices may include devices that are configured specifically for use with hearing instruments 102. Example types of accessory devices may include charging cases for hearing instruments 102, storage cases for hearing instruments 102, media streamer devices, phone streamer devices, external microphone devices, external telecoil devices, remote controls for hearing instruments 102, and other types of devices specifically designed for use with hearing instruments 102. Hearing instruments 102 and local computing system 106 may be configured to communicate with one another. In some examples, hearing instruments 102 may communicate with local computing system 106 using a BLUETOOTH technology. In some examples, an application running on local computing system 106 may allow users (e.g., user 104) to control and customize hearing instruments 102.

Remote computing system 108 may be remote from user 104. Local computing system 106 may communicate with remote computing system 108 via a communication network, such as the internet. In general, hearing instruments 102 do not communicate directly with remote computing system 108. In some examples, remote computing system 108 is a cloud-based computing system. Remote computing system 108 may include one or more computing devices, such as server devices.

In accordance with techniques of this disclosure, an artificial intelligence (AI)-enhanced virtual personal assistant 110 is provided to user 104 via hearing instruments 102. Virtual personal assistant 110 may help user 104 perform activities of daily living, such as providing reminders regarding meetings, appointments, or medications, providing reminders of past interactions with individual people, and so on. In some examples, virtual personal assistant 110 may help user 104 control and/or tune hearing instruments 102. In some examples, virtual personal assistant 110 may perform telehealth data collection.

Hearing instruments 102, local computing system 106 and/or remote computing system 108 may work together to provide virtual personal assistant 110. For example, microphones of hearing instruments 102 may detect speech and generate audio data, communication units of one or more of hearing instruments 102 may transmit the audio data to local computing system 106. Processors of hearing instruments 102 may preprocess the audio data. Processors of local computing system 106 and/or remote computing system 108 may further process the audio data and perform processing functions of virtual personal assistant 110. For instance, in some examples, remote computing system 108 may perform natural language processing (NLP), process voice commands, facilitate adjustments to hearing instruments 102, perform updates and diagnostics on hearing instruments 102, or perform other functionality of a virtual personal assistant 110. NLP may include speech-to-text, determining intention of speech requests, and otherwise extracting semantic content from natural language expressions. Local computing system 106 may transmit audio data, or semantic content that processors of one or more of hearing instruments 102 convert to audio data) to hearing instruments 102. Receivers of hearing instruments 102 may output auditory stimuli (e.g., sound) based on the audio data. In different examples of this disclosure, the processing functions of virtual personal assistant 110 may be distributed among processors of hearing instruments 102, local computing system 106, and remote computing system 108 in different ways.

User 104 may initiate interactions with virtual personal assistant 110, e.g., by speaking an activation word or phrase, pushing a button on one or more of hearing instruments 102, providing a command via local computing system 106, or performing some other action. In some examples, virtual personal assistant 110 may initiate an interaction with user 104 without user 104 explicitly initiating the interaction. In other words, in some examples, virtual personal assistant 110 does not need to wait for user 104 to initiate an interaction with virtual personal assistant 110. For instance, virtual personal assistant 110 may initiate an interaction with user 104 to provide reminders to user 104, offer help to user 104, and so on.

The use of hearing instruments 102 as an interface for interacting with virtual personal assistant 110 may have several benefits. For example, users tend to wear hearing instruments 102 almost all the time during their waking hours. This allows user 104 to have more opportunities to interact with virtual personal assistant 110 during most of their waking hours. Moreover, because users tend to wear hearing instruments 102 for prolonged periods, interacting with virtual personal assistant 110 via hearing instruments 102 may be a more seamless experience for users than trying to find a separate device, such as a smartphone or smart speaker. Additionally, because users tend to wear hearing instruments 102 for prolonged periods of time, hearing instruments 102 may be able to capture information that gives a more complete understanding of the user's activities, health, and personal interactions. Such information may include speech information, environmental information, health information, acoustic information, and so on.

Additionally, hearing instruments are uniquely capable of detecting and processing the speech of users of the hearing instruments. For instance, because hearing instruments 102 are placed in or near the ears of user 104, on either side of the vocal passage of user 104, hearing instruments 102 are well situated to distinguish the voice of user 104 from other the voices of other people. Additionally, hearing instruments 102, unlike other types of devices, may be tuned to overcome the specific hearing difficulties of user 104. This may enhance the ability of user 104 to naturally hear and understand virtual personal assistant 110.

Furthermore, hearing instruments 102 may be uniquely situated to collect relevant data about user 104 that may enhance the ability of virtual personal assistant 110 to interact with user 104. For instance, hearing instruments 102 may be well-situated to detect various health metrics of user 104, such as heart rate, body temperature, respiration rate, activity levels, detection of falls, galvanic skin response, and so on. Virtual personal assistant 110 may use or collect such data. In some examples, hearing instruments 102 may collect data (e.g., audio data, health data, activity data, and/or other types of data) throughout the time user 104 is wearing hearing instruments 102. In some examples, hearing instruments 102 may only collect data during specific times, in response to specific events, or data collection may be otherwise more limited in terms of times and situations.

As part of their role in compensating for hearing difficulties of users, hearing instruments 102 may perform various signal processing activities to improve the intelligibility of the audio data generated by microphones (e.g., microphones of hearing instruments 102, remote microphones, etc.). For example, hearing instrument 102 may perform signal processing to suppress wind noise, suppress background noise, perform directional beam processing to enhance sounds from specific directions, enhance human speech, and so on. Hearing instruments 102 may perform one or more of these same signal processing activities to preprocess the audio data used as a basis for interacting with virtual personal assistant 110. Thus, it may be unnecessary for such signal processing activities to be replicated at a separate computing system, which may reduce the overall complexity and cost of implementing virtual personal assistant 110. Additionally, because hearing instruments 102 may include processing circuitry specifically designed for such signal processing (because such specifically designed processing circuitry may be needed to support the hearing assistance role of hearing instruments 102), the signal processing may be faster than if implemented on more generic processors. Furthermore, the processed audio data may include less data than unprocessed data (e.g., due to filtering out background noise), which may conserve bandwidth and prolong battery life of hearing instruments 102 (and, in some instances, local computing system 106).

As mentioned briefly above, virtual personal assistant 110 may provide reminders to user 104 and help user 104 accomplish their daily activities. For example, virtual personal assistant 110 may learn and track a daily routine of user 104 based on information generated by hearing instruments 102 (and, in some examples, other sources). Parts of the daily routine may include eating, using the bathroom, showering/bathing, taking medications, exercising, watching television, and so on. Virtual personal assistant 110 may generate reminders if user 104 did not perform a specific task (e.g., taking medication, showering/bathing, eating, etc.). In some examples, because virtual personal assistant 110 may learn and track the daily routine of user 104, user 104 may ask virtual personal assistant 110 whether user 104 performed an activity. For instance, user 104 may ask virtual personal assistant 110 whether user 104 took their pills this morning, and virtual personal assistant 110 may provide a vocal response, such as “Yes, I heard you taking your pills this morning.” In some examples, virtual personal assistant 110 can track the remaining quantity of medication available to user 104 and remind user 104 to refill a prescription of the medication or may automatically request a refill of the prescription. Virtual personal assistant 110 may track the remaining quantity of medication based on spoken information provided to virtual personal assistant 110, a priori knowledge of medication dosage and provided quantities, or other sources. For instance, virtual personal assistant 110 may provide a vocal indication such as “I heard you saying you need to refill your prescription as you only had 2 pills left.”

In some examples, virtual personal assistant 110 has access to a calendar and may use the calendar to provide reminders to user 104. For example, virtual personal assistant 110 may remind user 104 about an upcoming appointment, social engagement, airtime of a favorite television show, mealtime, or other event. In some examples, the calendar may be shared among user 104 and other individuals, such as family members, community members, and caregivers. Thus, people other than user 104 may be able to add events to the calendar. Reminders about events may help users, especially those with memory impairments, live better lives and experience less frustration. In some examples, virtual personal assistant 110 may add events to the calendar based on audio data (which may or may not be explicitly directed to virtual personal assistant 110) generated by hearing instruments 102. For example, virtual personal assistant 110 may receive audio data indicating that user 102 has a doctor appointment at 3 pm on November 2. Accordingly, in this example, virtual personal assistant 110 may provide a reminder to user 104 about the doctor appointment at an appropriate time before the appointment.

In some examples, virtual personal assistant 110 may receive audio data generated by hearing instruments 102 representing the voices of people with whom user 104 is interacting. Based on audio data generated by hearing instruments 102, virtual personal assistant 110 may identify a person with whom user 104 is interacting. Virtual personal assistant 110 may learn and store information about the person (e.g., their relationship with user 104, content of interactions between the person and user 104, the person's name, the person's interests, etc.). Virtual personal assistant 110 may learn the information based on audio data generated by hearing instruments 102 and/or other sources. Virtual personal assistant 110 may use the information about the person with whom user 104 is interacting (or a person with whom user 104 may soon interact) to provide reminders to user 104 about the person. For instance, virtual personal assistant 110 may remind user 104 about the person's name, when user 104 last interacted with the person, what the person and user 104 have previously discussed, and provide other information about the person to user 104. Such reminders may be particularly helpful if user 104 has memory issues, face-blindness, or interacts with a large number of people.

As mentioned above, virtual personal assistant 110 may learn information about other people based on audio data generated by hearing instruments 102 and/or other sources. For instance, a user interface may be provided (e.g., by local computing system 106 and/or remote computing system 108) that enables people to provide information about themselves. In some examples, the user interface may allow people to provide voice samples. This may enhance the ability of virtual personal assistant 110 to provide information about people to user 104.

In some examples, user 104 may use virtual personal assistant 110 to control various aspects of hearing instruments 102. For example, user 104 may issue spoken commands to virtual personal assistant 110 to change the volume (e.g., global gain, shift gain profile against a range of frequencies, etc.) of hearing instruments 102 up or down. In some examples, user 104 may issue spoken commands to virtual personal assistant 110 to change a profile of hearing instruments 102 to restaurant mode, music listening mode, quiet mode, conversation mode, and so on. In some examples, user 104 may issue spoken commands to virtual personal assistant 110 to activate or deactivate features of hearing instruments 102, such as tinnitus masking, directional sound processing, noise suppression, remote microphones, and so on. In some examples, virtual personal assistant 110 may accept input to control aspects of hearing instruments 102 from sources other than audio data generated by hearing instruments 102, such as a user interface of a computing device used by user 104, a hearing professional, a caregiver, or another type of authorized person.

Furthermore, in some examples, virtual personal assistant 110 may store (e.g., in AI personalization data 410) data indicating that the commands of the user 104 to control or otherwise adjust the various settings of hearing instruments 102. In some such examples, virtual personal assistant 110 may also store contextual information along with the commands. The contextual information may include information related to an acoustic environment of hearing instruments 102, whether individual modes of hearing instruments 102 are activated or deactivated when user 104 issued the commands, physical locations or activities when user 104 issued the commands, and so on. In addition, virtual personal assistant 110 may store information indicating whether user 104 was satisfied with the settings of hearing instruments 102 after issuing the commands. For instance, if virtual personal assistant 110 receives subsequent commands while the contextual information remains substantially the same, virtual personal assistant 110 may store data indicating that user 104 was not satisfied with the adjusted settings of hearing instruments 102. However, if user 104 does not issue further commands, virtual personal assistant 110 may store information indicating that user 104 was satisfied with the adjusted settings. Virtual personal assistant 110 may subsequently use the stored information as a basis for suggesting potential solutions to complaints of user 104. Virtual personal assistant 110 may search the stored information for potential solutions to a problem described by user 104. For example, user 104 may initially issue a series of commands to adjust settings of hearing instruments 102 while hearing instruments 102 are in a loud restaurant environment. In this example, virtual personal assistant 110 may subsequently receive a request from user 104 to change the settings of hearing instruments 102 so that user 104 can hear better in a loud restaurant environment. In response, virtual personal assistant 110 may search the stored data, identify the settings with which user 104 was satisfied for the loud restaurant environment, and provide the settings as a proposed solution to user 104 wanting to hear better in the loud restaurant environment.

Virtual personal assistant 110 may allow user 104 to control aspects of hearing instruments 102 using a conversational style. For instance, user 104 may tell virtual personal assistant 110 that a specific type of sound (e.g., running water, rustling paper, etc.) does not sound right (e.g., is too loud, distorted, etc.) and virtual personal assistant 110 may determine an appropriate adjustment to one or more aspects of hearing instruments 102 to address the user's complaint. In some examples, user 104 may ask open ended questions to virtual personal assistant 110, to which virtual personal assistant 110 may make suggestions to change one or more aspects of hearing instruments 102 or automatically make changes to one or more aspects of hearing instruments 102. For example, virtual personal assistant 110 may receive and respond to an open-ended request or question regarding improving sound quality, e.g., “how should I improve my sound quality?” In responding to a request to improve sound quality, virtual personal assistant 110 may take into consideration various factors, such as environmental factors (e.g., noise levels), user history, user listening intent (e.g., comfort vs. clarity), and/or other factors. In other words, virtual personal assistant 110 may determine, based on such factors, one or more actions to adjust one or more aspects of hearing instruments 102. Virtual personal assistant 110 may determine environmental factors based on audio data generated by hearing instruments 102, which a computing system (e.g., hearing instruments 102, local computing system 106 or remote computing system 108) may store in a rolling buffer. Virtual personal assistant 110 may utilize a history of adjustments that user 104 has made in the past in different acoustic situations as a way to adjust aspects of hearing instruments 102.

In some examples, generative AI system 408 may use LLM 409 to predict garbled words in audio data from user 104. For example, one or more words in a spoken phrase may be unintelligible, e.g., due to wind noise, poor articulation, or other causes. In such examples, generative AI system 408 may provide a prompt to LLM 409 to request LLM 409 to automatically replace the garbled words. Generative AI system 408 may generate a prompt based on the spoken phrase with the replacement words. This may help reduce user frustration when working with virtual personal assistant 110.

In some examples, virtual personal assistant 110 may suggest and/or make adjustments to aspects of hearing instruments 102 based on the person or type of person with whom user 104 is talking. For instance, virtual personal assistant 110 may make adjustments to one or more aspects of hearing instruments 102 based on whether user 104 is speaking with a man, woman, or child, e.g., to improve speech intelligibility for user 104. In some examples, virtual personal assistant 110 may detect that the volume level of the person with whom user 104 is speaking is too low and may suggest increasing (or may automatically increase) gain.

Virtual personal assistant 110 may store a history of adjustments, requests for adjustments, and other factors. A hearing professional may use this history when determining how to manually adjust aspects of hearing instruments 102.

In some examples, virtual personal assistant 110 may use a trained machine learning (ML) model to predict adjustments that a hearing professional would make based on the user's statements and actions. The trained ML model may be trained based on data (e.g., listening environments, feedback from user 104, feedback from a population of users).

In some examples, when virtual personal assistant 110 receives a request to adjust one or more aspects of hearing instruments 102, virtual personal assistant 110 may provide an audible response to user 104, e.g., via one or more of hearing instruments 102, via local computing system 106 (e.g., a smartphone of user 104). The audible response may let user 104 know that virtual personal assistant 110 has made a change to the one or more aspects of hearing instruments 102. For instance, virtual personal assistant 110 may cause hearing instruments 102 to output a verbal response such as “Okay, let's try this” or a musical response (e.g., a “ta-da!” sound). In some examples, virtual personal assistant 110 may cause local computing system 106 (e.g., a smartphone of user 104) to provide a graphical or haptic indication that virtual personal assistant 110 has made a change to the one or more aspects of hearing instruments 102.

In some examples, virtual personal assistant 110 may perform an auto-fitting process to adjust aspects (e.g., global gain levels, frequency-specific gain levels, etc.) of hearing instruments 102 in response to a request from user 104 or other event. The auto-fitting process may involve hearing instruments 102 outputting specific tones and receiving responses from user 104 about the user's perception of the tones. Virtual personal assistant 110 may determine how to adjust aspects of hearing instruments 102 based on the responses from user 104. Virtual personal assistant 110 may use a set of predefined rules based on the user's responses to determine how to adjust the aspects of hearing instruments 102. In some examples, virtual personal assistant 110 may guide user 104 step-by-step through a self-fit process that helps user 104 adjust aspects of hearing instruments 102 to the personal needs and preferences of user 104.

In some examples, virtual personal assistant 110 may interact with user 104 to perform telehealth activities. For example, virtual personal assistant 110 may interact with user 104 to perform routine (e.g., daily, weekly, etc.) health checks. In this example, virtual personal assistant 110 may inquire how user 104 is feeling, inquire about specific symptoms, inquire about aspects of the mental and/or social health of user 104, and so on. Virtual personal assistant 110 may aggregate the responses user 104 to the health checks to form a longer term understanding of the physical and/or mental well-being of user 104.

In some examples, virtual personal assistant 110 may detect changes in one or more health indicators of user 104 and respond accordingly. For instance, virtual personal assistant 110 may detect changes in the speech of user 104 that may be indicative of a stroke, cognitive decline (e.g., pausing more, phrases/sounds indicative of memory recall problems, declining vocabulary set, etc.), agitation, and so on. In some examples, virtual personal assistant 110 may detect changes to the gait of user 104 based on motion signals generated by motion sensors of hearing instruments 102.

In some examples, virtual personal assistant 110 may use LLM 409 to generate sociability data for user 104. The sociability data may provide information about the sociability of user 104. For instance, the sociability data may indicate how frequently user 104 interacts with other people, how frequently user 104 interacts with new people, the number of people with which user 104 interacts, and so on. LLM 409 may use data collected from hearing instruments 102 to generate the sociability data. For instance, generative AI system 408 may provide a prompt to LLM 409 that includes own-voice data, anonymized information that identifies conversation partners, and so. The prompt may request LLM 409 to describe the sociability of user 104. Virtual personal assistant 110 may provide the description to user 104 or another person.

In some examples, user 104 may use virtual personal assistant 110 without hearing instruments 102. In some such examples, virtual personal assistant 110 may simulate for user 104 what a sound would be like with hearing loss, or with a hearing aid.

In some examples, virtual personal assistant 110 may receive and respond to questions from user 104 regarding hearing instruments 102. For instance, virtual personal assistant 110 may receive and respond to help requests from user 104. Other types of questions may include questions about hearing instruments 102 themselves, such as the type of battery, model information for hearing instruments, battery levels of hearing instruments, hearing instrument usage times, how hearing instruments 102 work, how to use features of hearing instruments 102, troubleshoot problems with hearing instruments 102, and so on.

Virtual personal assistant 110 may use generative AI techniques to generate responses to user 104. For instance, virtual personal assistant 110 may use a large language model (LLM) to generate responses. Examples of LLMs include ChatGPT by OpenAI, LLaMA by Meta Platforms, Inc. PaLM from Google, Inc., and so on. Thus, in some such examples, virtual personal assistant 110 may present information (e.g., text of a request of user 104, data regarding environmental acoustic factors, user history, etc.) as a prompt to the LLM. The LLM may then generate a response (e.g., a textual response). Depending on the prompt, the LLM may generate different types of responses. For example, a request to related to changing one or more aspects of hearing instruments 102, may describe a series of actions or steps, such as actions to adjust one or more aspects of hearing instruments 102. In another example, a prompt may request information about a conversation partner may cause the LLM to generate a query to retrieve information about the conversation partner from a database and use the retrieved information as another prompt to cause the LLM to format the retrieved information in a conversational style.

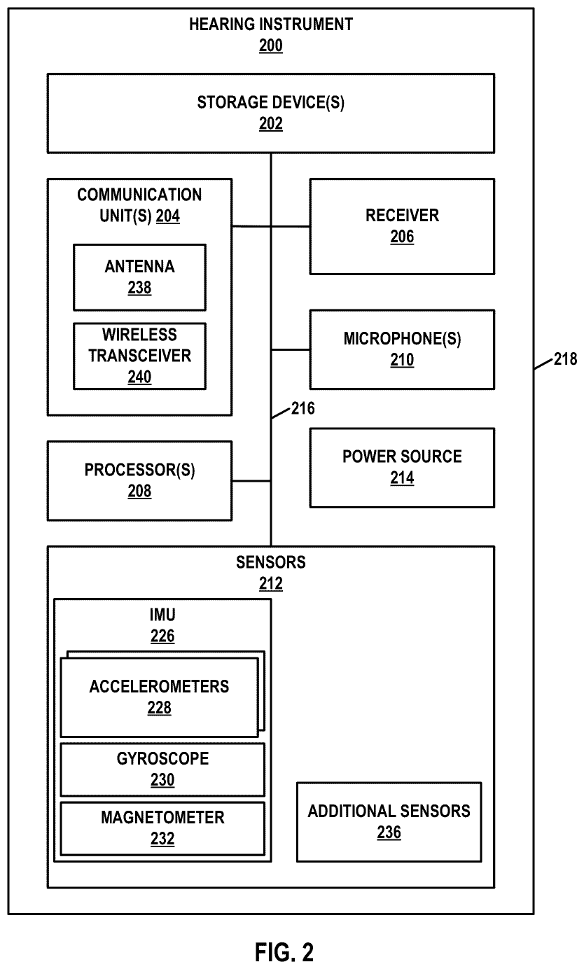

FIG. 2 is a block diagram illustrating example components of hearing instrument 102A, in accordance with one or more aspects of this disclosure. Hearing instrument 102B may include the same or similar components of hearing instrument 102A shown in the example of FIG. 2. Thus, the discussion of FIG. 2 may apply with respect to hearing instrument 102B. In the example of FIG. 2, hearing instrument 102A includes one or more storage devices 202, one or more communication units 204, a receiver 206, one or more processors 208, one or more microphones 210, a set of sensors 212, a power source 214, and one or more communication channels 216. Communication channels 216 provide communication between storage devices 202, communication unit(s) 204, receiver 206, processor(s) 208, microphone(s) 210, and sensors 212. Components 202, 204, 206, 208, 210, 212, and 216 may draw electrical power from power source 214.

In the example of FIG. 2, each of components 202, 204, 206, 208, 210, 212, 214, and 216 are contained within a single housing 218. For instance, in examples where hearing instrument 102A is a BTE device, each of components 202, 204, 206, 208, 210, 212, 214, and 216 may be contained within a behind-the-ear housing. In examples where hearing instrument 102A is an ITE, ITC, CIC, or IIC device, each of components 202, 204, 206, 208, 210, 212, 214, and 216 may be contained within an in-ear housing. However, in other examples of this disclosure, components 202, 204, 206, 208, 210, 212, 214, and 216 are distributed among two or more housings. For instance, in an example where hearing instrument 102A is a RIC device, receiver 206, one or more of microphones 210, and one or more of sensors 212 may be included in an in-ear housing separate from a behind-the-ear housing that contains the remaining components of hearing instrument 102A. In such examples, a RIC cable may connect the two housings.

Furthermore, in the example of FIG. 2, sensors 212 include an inertial measurement unit (IMU) 226 that is configured to generate data regarding the motion of hearing instrument 102A. IMU 226 may include a set of sensors. For instance, in the example of FIG. 2, IMU 226 includes one or more accelerometers 228, a gyroscope 230, a magnetometer 232, combinations thereof, and/or other sensors for determining the motion of hearing instrument 102A. Furthermore, in the example of FIG. 2, hearing instrument 102A may include one or more additional sensors 236. Additional sensors 236 may include a photoplethysmography (PPG) sensor, blood oximetry sensors, blood pressure sensors, electrocardiograph (EKG) sensors, body temperature sensors, electroencephalography (EEG) sensors, environmental temperature sensors, environmental pressure sensors, environmental humidity sensors, skin galvanic response sensors, and/or other types of sensors. In other examples, hearing instrument 102A and sensors 212 may include more, fewer, or different components.

Storage device(s) 202 may store data. Storage device(s) 202 may include volatile memory and may therefore not retain stored contents if powered off. Examples of volatile memories may include random access memories (RAM), dynamic random access memories (DRAM), static random access memories (SRAM), and other forms of volatile memories known in the art. Storage device(s) 202 may include non-volatile memory for long-term storage of information and may retain information after power on/off cycles. Examples of non-volatile memory may include flash memories or forms of electrically programmable memories (EPROM) or electrically erasable and programmable (EEPROM) memories.

Communication unit(s) 204 may enable hearing instrument 102A to send data to and receive data from one or more other devices, such as a device of computing system 106 (FIG. 1), another hearing instrument (e.g., hearing instrument 102B), an accessory device, a mobile device, or other types of devices. Communication unit(s) 204 may enable hearing instrument 102A to use wireless or non-wireless communication technologies. For instance, communication unit(s) 204 enable hearing instrument 102A to communicate using one or more of various types of wireless technology, such as a BLUETOOTH™ technology, 3G, 4G, 4G LTE, 5G, ZigBee, WI-FI™, Near-Field Magnetic Induction (NFMI), ultrasonic communication, infrared (IR) communication, or another wireless communication technology. In some examples, communication unit(s) 204 may enable hearing instrument 102A to communicate using a cable-based technology, such as a Universal Serial Bus (USB) technology.

Receiver 206 includes one or more speakers for generating auditory stimuli, such as audible sound, vibration, or cochlear stimulation signals. In the example of FIG. 2, receiver 206 includes speaker 108A (FIG. 1). The speakers of receiver 206 may generate auditory stimuli that include a range of frequencies. In some examples, the speakers of receiver 206 includes “woofers” and/or “tweeters” that provide additional frequency range.

Processor(s) 208 include processing circuits configured to perform various processing activities. Processor(s) 208 may process signals generated by microphone(s) 210 to enhance, amplify, or cancel-out particular channels within the incoming sound. Processor(s) 208 may then cause receiver 206 to generate auditory stimuli based on the processed signals. In some examples, processor(s) 208 include one or more digital signal processors (DSPs). In some examples, processor(s) 208 may cause communication unit(s) 204 to transmit one or more of various types of data. For example, processor(s) 208 may cause communication unit(s) 204 to transmit data to computing system 106. Furthermore, communication unit(s) 204 may receive audio data from local computing system 106 and processor(s) 208 may cause receiver 206 to output auditory stimuli based on the audio data. In the example of FIG. 2, processor(s) 208 include processors 112A (FIG. 1).

Microphone(s) 210 detect incoming sound and generate one or more electrical signals (e.g., an analog or digital electrical signal) representing the incoming sound. In some examples, microphone(s) 210 include directional and/or omnidirectional microphones.

In accordance with one or more techniques of this disclosure, communication unit(s) 204 may send audio data (and, in some examples other data, such as sensor data) to local computing system 106 for eventual processing by virtual personal assistant 110. In some examples, processors 208 may process audio data generated by microphone(s) 210 prior to communication unit(s) 204 transmitting the audio data. As previously discussed, preprocessing the audio data in this way may be efficient because hearing instrument 200 may already be equipped for such audio processing. Additionally, communication unit(s) 204 may receive an output from virtual personal assistant 110 (e.g., audio data or other types of data from local computing system 106). Processor(s) 208 may convert the output into an audio signal that receiver 206 may convert into auditory stimuli.

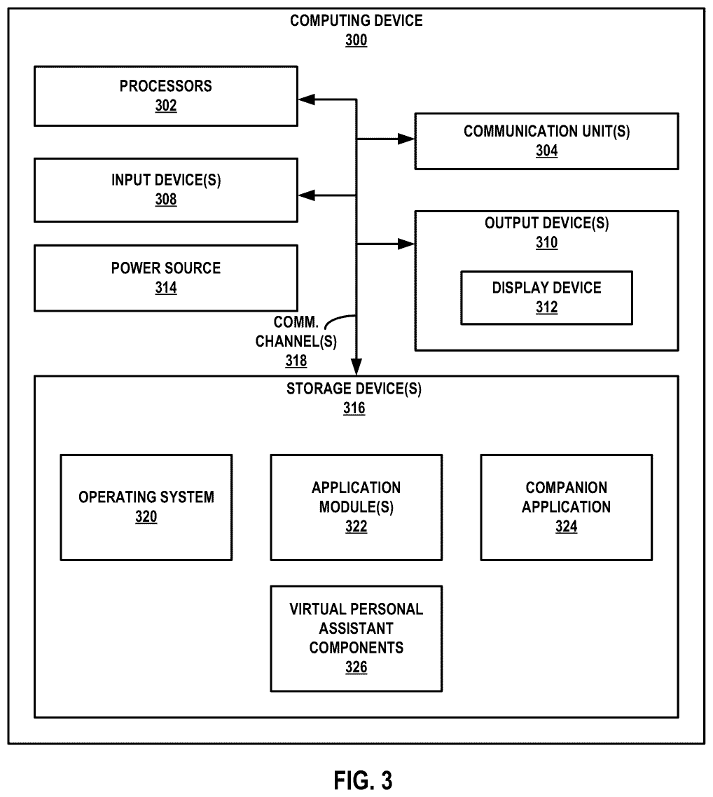

FIG. 3 is a block diagram illustrating example components of a computing device 300, in accordance with one or more aspects of this disclosure. FIG. 3 illustrates only one example of computing device 300, and many other example configurations of computing device 300 exist. Computing device 300 may be a computing device in local computing system 106 or remote computing system 108 (FIG. 1).

As shown in the example of FIG. 3, computing device 300 includes one or more processors 302, one or more communication units 304, one or more input devices 308, one or more output device(s) 310, a display screen 312, a power source 314, one or more storage device(s) 316, and one or more communication channels 318. Computing device 300 may include other components. For example, computing device 300 may include physical buttons, microphones, speakers, communication ports, and so on. Communication channel(s) 318 may interconnect each of components 302, 304, 308, 310, 312, and 316 for inter-component communications (physically, communicatively, and/or operatively). In some examples, communication channel(s) 318 may include a system bus, a network connection, an inter-process communication data structure, or any other method for communicating data. Power source 314 may provide electrical energy to components 302, 304, 308, 310, 312 and 316.

Storage device(s) 316 may store information required for use during operation of computing device 300. In some examples, storage device(s) 316 have the primary purpose of being a short-term and not a long-term computer-readable storage medium. Storage device(s) 316 may include volatile memory and may therefore not retain stored contents if powered off. In some examples, storage device(s) 316 includes non-volatile memory that is configured for long-term storage of information and for retaining information after power on/off cycles. In some examples, processor(s) 302 of computing device 300 may read and execute instructions stored by storage device(s) 316.

Computing device 300 may include one or more input devices 308 that computing device 300 uses to receive user input. Examples of user input include tactile, audio, and video user input. Input device(s) 308 may include presence-sensitive screens, touch-sensitive screens, mice, keyboards, voice responsive systems, microphones, motion sensors capable of detecting gestures, or other types of devices for detecting input from a human or machine.

Communication unit(s) 304 may enable computing device 300 to send data to and receive data from one or more other computing devices (e.g., via a communication network, such as a local area network or the Internet). For instance, communication unit(s) 304 may be configured to receive data sent by hearing instrument(s) 102, receive data generated by user 104 of hearing instrument(s) 102, receive and send data, receive and send messages, and so on. In some examples, communication unit(s) 304 may include wireless transmitters and receivers that enable computing device 300 to communicate wirelessly with the other computing devices. For instance, in the example of FIG. 3, communication unit(s) 304 include a radio 306 that enables computing device 300 to communicate wirelessly with other computing devices, such as hearing instruments 102 (FIG. 1). Examples of communication unit(s) 304 may include network interface cards, Ethernet cards, optical transceivers, radio frequency transceivers, or other types of devices that are able to send and receive information. Other examples of such communication units may include BLUETOOTH™, 3G, 4G, 5G, and WI-FI™ radios, Universal Serial Bus (USB) interfaces, etc. Computing device 300 may use communication unit(s) 304 to communicate with one or more hearing instruments (e.g., hearing instruments 102 (FIG. 1, FIG. 2)). Additionally, computing device 300 may use communication unit(s) 304 to communicate with one or more other devices.

Output device(s) 310 may generate output. Examples of output include tactile, audio, and video output. Output device(s) 310 may include presence-sensitive screens, sound cards, video graphics adapter cards, speakers, liquid crystal displays (LCD), light emitting diode (LED) displays, or other types of devices for generating output. Output device(s) 310 may include display screen 312. In some examples, output device(s) 310 may include virtual reality, augmented reality, or mixed reality display devices.

Processor(s) 302 may read instructions from storage device(s) 316 and may execute instructions stored by storage device(s) 316. Execution of the instructions by processor(s) 302 may configure or cause computing device 300 to provide at least some of the functionality ascribed in this disclosure to computing device 300 or components thereof (e.g., processor(s) 302). As shown in the example of FIG. 3, storage device(s) 316 include computer-readable instructions associated with operating system 320, application modules 322A-322N (collectively, “application modules 322”), a companion application 324, and one or more virtual personal assistant components 326. In examples where computing device 300 is part of remote computing system 106, application models 322 and companion application 324 (along with some hardware components, such as input devices 308 and/or output devices 310) may be omitted.

Execution of instructions associated with operating system 320 may cause computing device 300 to perform various functions to manage hardware resources of computing device 300 and to provide various common services for other computer programs. Execution of instructions associated with application modules 322 may cause computing device 300 to provide one or more of various applications (e.g., “apps,” operating system applications, etc.). Application modules 322 may provide applications, such as text messaging (e.g., SMS) applications, instant messaging applications, email applications, social media applications, text composition applications, and so on.

Companion application 324 is an application that may be used (e.g., by user 104 or another person) to interact with hearing instruments 102, view information about hearing instruments 102, or perform other activities related to hearing instruments 102. Execution of instructions associated with companion application 324 by processor(s) 302 may cause computing device 300 to perform one or more of various functions. For example, execution of instructions associated with companion application 324 may cause computing device 300 to configure communication unit(s) 304 to receive data from hearing instruments 102 and use the received data to present data to a user, such as user 104 or a third-party user. For instance, companion application 324 may be used to provide calendar information, voice sample information, and so on. In some examples, companion application 324 is an instance of a web application or server application. In some examples, such as examples where computing device 300 is a mobile device or other type of computing device, companion application 324 may be a native application.

Virtual personal assistant components 326 may perform some or all tasks of virtual personal assistant 110. Virtual personal assistant components 326 may be distributed and/or replicated among multiple computing devices, including devices of local computing system 106 and remote computing system 108.

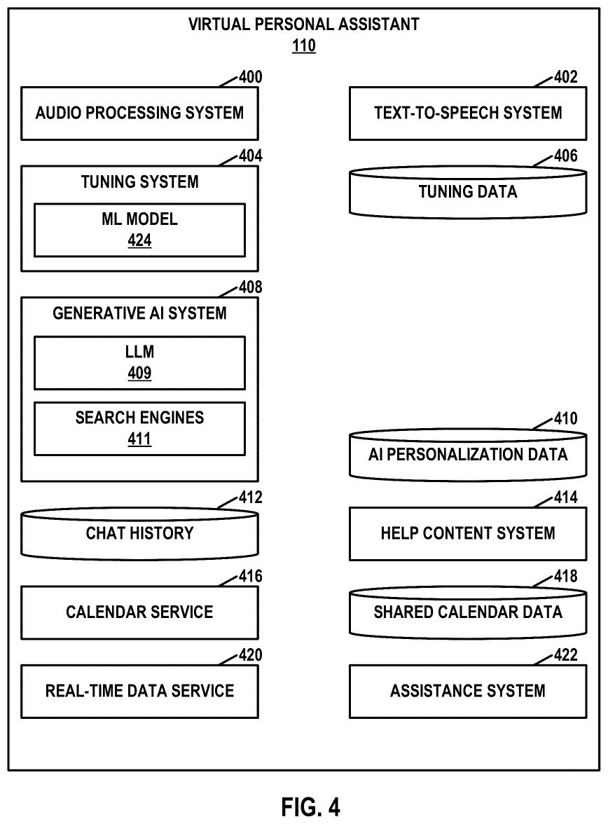

FIG. 4 is a block diagram illustrating example components of virtual personal assistant 110, in accordance with one or more techniques of this disclosure. The components of virtual personal assistant 110 may be within a single computing device or may be distributed among two or more devices, including hearing instruments 102, local computing system 106, and remote computing system 108.

In the example of FIG. 4, virtual personal assistant 110 includes an audio processing system 400, a text-to-speech system 402, a tuning system 404, tuning data 406, a generative AI system 408, AI personalization data 410, a chat history 412, a help content system 414, a calendar service 416, shared calendar data 418, a real-time data service 420, and an assistance system 422. Audio processing system 400 may receive audio data and perform natural language processing to determine the semantic content of speech in the audio data. In some examples, audio processing system 400 may process the audio data to obtain other information, such as information about acoustic conditions. Text-to-speech system 402 may convert text to audio data representing speech.

Tuning system 404 may suggest adjustments (or may automatically adjust aspects of hearing instruments 102. Tuning data 406 may include data regarding adjustments to hearing instruments 102. Tuning system 404 may use tuning data 406 to suggest or make adjustments to one or more aspects of hearing instruments 102. In some examples, tuning system 404 may use a trained ML model 424 to predict adjustments to the one or more aspects of hearing instruments 102. ML model 424 may be trained based on adjustment histories from user 104 and/or a population of users, including tuning data 406.

Generative AI system 408 may include a LLM 409 that generates textual responses to prompts. AI personalization data 410 may include data that generative AI system 408 may use to personalize responses to user 104. For instance, AI personalization data 410 may include tuning data 406, user personalization data, insights, recommendations, and so on. Chat history 412 may include a history of interactions between user 104 and virtual personal assistant 110 that generative AI system 408 may use to generate responses.

Help content system 414 may generate response to requests from user 104 for help regarding hearing instruments 102. Calendar service 416 may use shared calendar data 418 to provide reminders and otherwise provide chronological data to user 104. Real-time data service 420 may retrieve information from live information sources (e.g., weather data, stock price data, news data, etc.) to provide to hearing instruments 102. Assistance system 422 may coordinate activities of other components of virtual personal assistant 110.

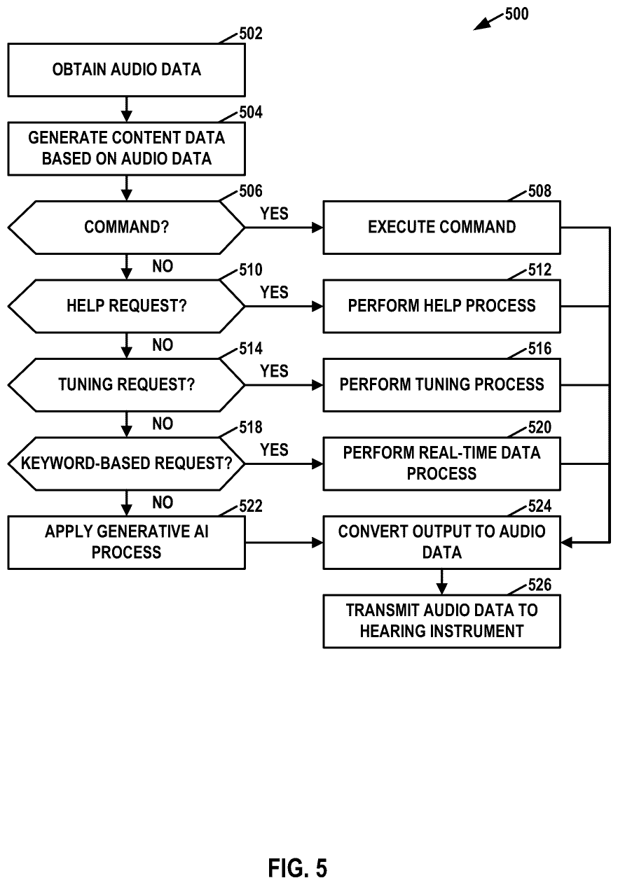

FIG. 5 is a flowchart illustrating an example operation 500 of virtual personal assistant 110 in accordance with one or more techniques of this disclosure. Other examples of this disclosure may include more, fewer, or different actions. In some examples, actions in the flowcharts of this disclosure may be performed in parallel or in different orders.

In the example of FIG. 5, audio processing system 400 may obtain audio data (502). For instance, audio processing system 400 may receive the audio data from one or more of hearing instruments 102. Audio processing system 400 may generate content data based on the audio data (504). For example, audio processing system 400 may perform speech recognition and natural language processing (NLP) to extract semantic content data from the audio data. In some examples, audio processing system 400 may extract non-speech data, such as data indicating acoustic conditions, from the audio data. Although not shown in the example of FIG. 5, virtual personal assistant 110 may also obtain other content data, such as motion data, sensor data, information indicating a caregiver of user 104, information indicating how long user 104 has had hearing instruments 102, environmental information (e.g., information describing a physical and/or acoustic environment of user 104, information regarding interactions between user 104 and an audiologist (e.g., audiologist notes, fitting session notes, etc.), sociability information regarding user 104, information regarding amounts of time user 104 has worn hearing instruments 102, and so on. Thus, in some examples, virtual personal assistant 110 may generate responses based in part on sensor data, such as sensor data generated by sensors of hearing instruments 102. Generating responses based in part on such sensor data may increase the accuracy or usefulness of the responses.

Assistance system 422 may determine, based on the content data, whether user 104 is providing a command to virtual personal assistant 110 (506). Example commands may include direct requests to change volume, turn on or off features, change acoustic programs, and so on. If user 104 is providing a command to virtual personal assistant 110 (“YES” branch of 506), assistance system 422 may send instructions to hearing instruments 102 to execute the command (508). Assistance system 422 may also generate or retrieve textual output data indicating a response to the command (e.g., “ok, turning up the volume”). Text-to-speech system 402 may convert the textual output data to audio data (524) and assistance system 422 may transmit the audio data to hearing instruments 102 (526).

Otherwise, if user 104 is not providing a command to virtual personal assistant 110 (“NO” branch of 506), assistance system 422 may determine whether the content data represents a help request (510). Example help requests may include requests for information about hearing instruments 102. If the content data represents a help request (“YES” branch of 510), help content system 414 may perform a help process to generate a help response (512). The help process may use a keyword-based search to retrieve predefined text that corresponds to the help request. Text-to-speech system 402 may convert textual output data of help content system 414 to audio data (524) and assistance system 422 may transmit the audio data to hearing instruments 102 (526).

If the content data does not represent a help request (“NO” branch of 510), assistance system 422 may determine whether the content data represents a tuning request (514). If assistance system 422 determines that the content data represents a tuning request (“YES” branch of 514), tuning system 404 may perform a tuning process to recommend or apply one or more adjustments to one or more aspects of hearing instruments 102 (516). In some examples, tuning system 404 may guide user 104 through a self-fit or auto-fit process. Text-to-speech system 402 may convert textual output data of the tuning process to audio data (524) and assistance system 422 may transmit the audio data to hearing instruments 102 (526). In some examples, actions (514) and (516) are not included in operation 500.

Assistance system 422 may determine whether the content data represents a keyword-based request (518). For instance, in the example of FIG. 5, assistance system 422 may determine whether the content data represents a keyword-based request in response to determining that the content data does not represent a tuning request. A keyword-based request may include one or more keywords that indicate that user 104 is seeking real-time information, such as news or weather information. If assistance system 422 determines that the content data represents a keyword-based request (“YES” branch of 518), real-time data service 420 may perform a real-time data process (520). Performance of the real-time data process may involve retrieval of information from online sources, such as webpages, application programming interfaces (APIs), and so on. Text-to-speech system 402 may convert textual output data of the real-time data process to audio data (524) and assistance system 422 may transmit the audio data to hearing instruments 102 (526).

If assistance system 422 determines that the content data does not represent a keyword-based request (“NO” branch of 518), generative AI system 408 may perform a generative AI process to generate a response (522). As part of performing the generative AI process, generative AI system 408 may apply an LLM one or more times to generate the response. In some examples, the response may be conversational in tone, similar to what a user might experience using a chatbot, such as ChatGPT, Google BARD, Google GEMINI, Microsoft COPILOT, and so on. In some examples, the response may be to add data to shared calendar data 418, AI personalization data 410, and so on. Chat history 412 may include a record of interactions of user 104 with virtual personal assistant 110, including records of interactions with generative AI system 408. In some examples, generative AI system 408 may automatically summarize conversations that user 104 had with other people, generate data indicating performance of activities, generate information regarding sociability of user 104, generate information regarding the patterns with which user 104 uses hearing instruments 102, create memories (i.e., combinations of settings of hearing instruments 102 associated with specific situations), generate recommendation for user 104 to consult an audiologist, generate information regarding progression of hearing loss and associated remediation actions, and so on, and store the resulting data as AI personalization data 410. Generative AI system 408 (or another component of virtual personal assistant 110) may use the stored data for various purposes, such as providing reminders, cognitive support, and so on. Thus, not all responses generated by generative AI system 408 are for immediate responses to user 104 and generative AI system 408 may generate a response (e.g., for internal data storage) without immediate user 104 involvement. In some examples, the generated information may be shared with individuals other than user 104, such as an audiologist, caregiver, family member, or other type of person. Text-to-speech system 402 may convert textual output data of the real-time data process to audio data (524) and assistance system 422 may transmit the audio data to hearing instruments 102 (526).

Assistance system 422 may determine whether the content data represents a command, help request, tuning request, keyword-based request, or other type of request based on keyword matching within the content data, based on a semantic analysis of the content data, or in another way. In some examples, calendar service 416 may operate outside of operation 500 so that calendar service 416 may provide reminders separate that are not in response to requests from user 104. Operation 500 may avoid use of generative AI system 408 for tasks that do not require complex processing. This may save computational resources.

In some examples, generative AI system 408 may receive content data that comprises a help request. For instance, generative AI system 408 may receive content data generated by audio processing system 400 based on audio data generated by one or more of hearing instruments 102. In some examples, companion application 324, a web application, or another application may receive input of textual input of the content data from user 104.

The help request may indicate that user 104 is seeking help with respect to one or more problems with hearing instruments 102. Example problems with hearing instruments 102 may include: all sounds are too loud, all sounds are too soft, distant sounds are fine but sounds that are close by are too soft, fan noises are louder than voices, background noises fluctuate or are pumping, loud sounds are too loud, my own voice does not sound normal, paper sounds are harsh or loud, the sound of keys rattling is too harsh or loud, the sound of dishes clattering is too loud, hearing speech in noisy environments is difficult, water sounds are harsh, tinny, or too loud, female or child voices are hard to understand, and so on.

Generative AI system 408 may generate a first prompt based on the help request. The first prompt may request that LLM 409 pretend to be a virtual personal assistant and to find a solution to the help request. In response to the first prompt, LLM 409 may generate one or more search requests (e.g., one or more queries). Each of the one or more search requests may comprise text representing a request to retrieve data items that satisfy criteria specified in the search request. For example, the help request may specify that user 104 is seeking a solution to a specific problem with hearing instruments 102, such as the sound of running water being disproportionately loud. In this example, the one or more search requests may request data items that describe solutions to the specific problem. Example solutions may include changing settings of hearing instruments 102, suggesting to user 104 to change or recharge a battery of one or more of hearing instruments 102, turning or disabling on edge mode, turning or disabling specific sets of settings (e.g., a set of settings for restaurant environments), changes to gain levels at specific frequency bands, and so on, suggesting running a self-check on one or more of hearing instruments 102, suggest upgrading hearing instruments 102, and so on. Edge mode is a mode in which hearing instruments 102 attempt to predict settings suitable for the needs of user 104.

One or more search engines 411 may use the one or more search requests to search one or more content repositories for applicable data items. The data items may include database entries, webpages, documents, articles, audiologist records, product manuals, or other types of stored data. Example search engines may include database management systems, web search engines, and so on. Although shown in FIG. 4 as being part of generative AI system 408, one or more of search engines 411 may be separate from generative AI system 408, virtual personal assistant 110, computing device 300, local computing system 106, or remote computing system 108. The search may be limited to specific content repositories, such as a database on hearing instrument help items, AI personalization data 410, tuning data 406, chat history 412, and so on. Thus, the applicable data items may include data indicating previous settings of hearing instruments 102, previous interactions between user 104 and virtual personal assistant 110, data items describing solutions that other people have identified for the help request, and so on. In some examples, the applicable data items include data items that are specific to user 104. In some examples, the applicable data items include data items that are not specific to user 104. In some examples, the applicable data items exclusive include data items that are not specific to user 104. When the content repositories include data items specific to users other than user 104, the search does not return data items corresponding to the other users. Thus, personal and/or personally identifiable information of the other users is not included in the returned applicable data items.

The one or more search engines 411 may provide the applicable data items to generative AI system 408. Thus, generative AI system 408 may obtain the applicable data items. Generative AI system 408 may generate a second prompt that includes the applicable data items. The second prompt may request LLM 409 to describe a solution to the help request based on the applicable data items. The second prompt may also specify that the virtual personal assistant should not provide one or more types of inappropriate responses. After generating the second prompt, generate AI system 408 may provide the second prompt to LLM 409. LLM 409 may generate a response based on the second prompt. The response may describe a solution to the help request. For example, if the help request indicates that user 104 was having difficulty hearing the voices of children, the response may specify potential changes to settings of hearing instruments 102 to alleviate this problem. In some examples, the response includes a textual description of how the user can make the changes to the settings of the hearing instruments 102. Text-to-speech system 402 may convert the textual description to audio data, which hearing instruments 102 may convert to sound. In some examples, the response includes computer-executable commands (e.g., application programming interface (API) requests) that instruct one or more of hearing instruments 102 to change one or more settings. In some such examples, the response may also include textual data to inform user 104 that virtual personal assistant 110 has identified a potential solution and to ask user 104 whether to apply the potential solution to hearing instruments 102. Virtual personal assistant 110 may apply the potential solution to hearing instruments 102 in response to receiving an indication that user 104 assents to applying the potential solution.

In some instances, LLM 409 may identify multiple potential solutions to the help request. In some such examples, LLM 409 may determine an order of the potential solutions by sorting the potential solutions based on a measure of how much each of the solutions changes the settings of hearing instruments 102. LLM 409 may provide responses based on the determined order. If generative AI system 408 receives content data indicating that user 104 is not satisfied when a potential solution is applied to hearing instruments 102, generative AI system 408 may generate a response based on the next potential solution of the determined order. In this way, changes to the settings of hearing instruments 102 may be made gradually, which may be less disconcerting to user 104 than sudden large changes to the settings of hearing instruments 102.

Virtual personal assistant 110 may store information indicating responses from user 104. For instance, virtual personal assistant 110 may store information indicating that a potential solution did or did not solve the help request. The stored information may form part of the data items that can be retrieved by the one or more search engines in response to search requests generated by LLM 409.

In some examples, virtual personal assistant 110 may proactively prompt user 104, via hearing instruments 102, to provide a response. For instance, virtual personal assistant 110 may periodically prompt LLM 409 to generate information about user 104, such as descriptions of user behavior, e.g., sociability, trouble with hearing instruments 102, etc. Virtual personal assistant 110 may also prompt LLM 409 to generate output that prompts user 104 with respect to the user behavior. For instance, the output may ask user 104 whether user 104 has been having trouble with hearing instruments 102 lately. In another example, the output may ask user 104 about their social interactions. In some examples, virtual personal assistant 110 may proactively identify potential changes to settings of hearing instruments 102 and prompt user 104 to indicate whether user 104 would like to change the one or more settings of hearing instruments 102, e.g., based on contextual information, such as information regarding an acoustic environment, activities of user 104, location data, sensor data, and so on.

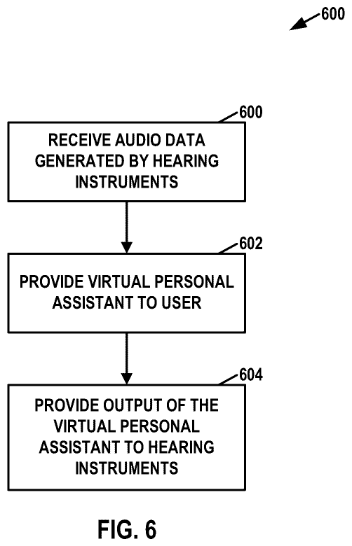

FIG. 6 is a flowchart illustrating an example operation 600, in accordance with one or more techniques of this disclosure. In the example of FIG. 6, a computing system (e.g., local computing system 106, remote computing system 108, and/or components of one or more of hearing instruments 102) may receive audio data generated by one or more hearing instruments 102 worn at or near one or more ears of user 104 (600). The audio data may be first audio data and hearing instruments 102 may be configured to generate the first audio data by applying signal processing to second audio data generated by microphones of the one or more hearing instruments. In some examples, audio processing system 400 may extract non-speech data, such as data indicating acoustic conditions, from the audio data. Computing system 108 may also obtain other content data, such as motion data, sensor data, information indicating a caregiver of user 104, information indicating how long user 104 has had hearing instruments 102, environmental information (e.g., information describing a physical and/or acoustic environment of user 104, information regarding interactions between user 104 and an audiologist (e.g., audiologist notes, fitting session notes, etc.), sociability information regarding user 104, information regarding amounts of time user 104 has worn hearing instruments 102, and so on.

The computing system may provide a virtual personal assistant 110 to user 104 (602). Virtual personal assistant 110 may be configured to generate, based on the audio data (and, in some examples other non-speech data), output to assist user 104. The computing system may provide the output to the one or more hearing instruments 102 (604). Hearing instruments 102 may be configured to generate auditory stimuli based on the output.

In some examples, as part of providing virtual personal assistant 110, the computing system applies a Large Language Model (LLM) to generate a response. The output of virtual personal assistant 110 may be based on the response. In some examples, providing virtual personal assistant 110 may further comprise generating a prompt based on the audio data and applying the LLM to the prompt to generate the response.

In some examples, virtual personal assistant 110 is configured to learn a routine of the user based at least in part on the audio data and generate the output based on the routine of the user. The output of virtual personal assistant 110 may be based on the routine of the user includes a reminder to perform an activity.

In some examples, virtual personal assistant 110 is configured to determine, based on the audio data, whether an event has occurred and to generate the output indicating whether the event has occurred. For instance, the event may be user 104 taking medication. In some examples, virtual personal assistant 110 is configured to access a calendar and the output is based on events in the calendar.

In some examples, the audio data represent a voice of a person with whom the user is interacting, and the output generated by the virtual personal assistant includes information about the person. In some such examples, the information about the person includes information about interactions between the person and the user. Virtual personal assistant 110 may be configured to learn the information about the person based on the audio data received from hearing instruments 102.

Furthermore, in some examples, the output generated by virtual personal assistant 110 includes a recommended or automatic adjustment to one or more aspects of the one or more hearing instruments. In some such examples, the audio data may include a request from the user to improve sound quality of the one or more hearing instruments. In some examples, virtual personal assistant 110 is configured to receive health data for the user. In some examples, virtual personal assistant 110 extracts semantic content of speech represented by the audio data.

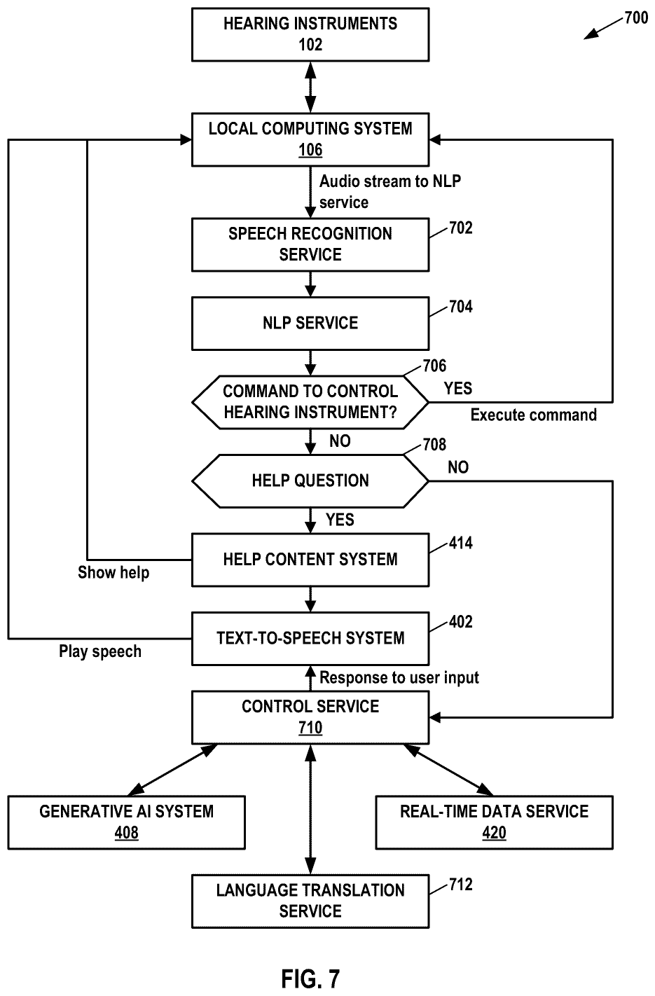

FIG. 7 is a conceptual diagram illustrating an example software architecture 700, in accordance with one or more techniques of this disclosure. In the example of FIG. 7, hearing instruments 102 communicate with local computing system 106. Hearing instruments 102 may transmit audio data to local computing system 106. The audio data may represent voice input from user 104. The voice input may include speech sounds of user 104. The audio data, including the voice input, may represent sound detected by one or more microphones (e.g., microphones 210) of one or more hearing instruments 102 and/or sound detected by one or more microphones of local computing system 106. In some examples, the audio data may also represent background noise, speech sounds of other people, and so on. Furthermore, in some examples, hearing instruments 102 may send data in addition to audio data to local computing system 106. For instance, hearing instruments 102 may send sensor data to local computing system 106. Thus, assistance system 422 may obtain the audio data.

Assistance system 422 may provide the audio data to speech recognition service 702. Speech recognition service 702 may be included in audio processing system 400 (FIG. 4). Speech recognition service 702 may process the audio data to convert speech. including voice input, represented by the audio data to text.

A natural language processing (NLP) service 704 may extract semantic content from the text. Audio NLP service 704 may also be included in audio processing system 400 (FIG. 4). For example, NLP service 704 may determine an intention of the user based on the text. An example intention may be to command one or more of hearing instruments 102 to perform one or more actions. Another example intention may be to request help with respect to one or more of hearing instruments 102. In some examples, NLP service 704 may determine that the intention is to execute a command based on the presence of one or more keywords in the voice input associated with executing a command. Similarly, NLP service 704 may determine that the intention is to request help based on the presence of one or more keywords in the voice input associated with requesting help.

If the voice input represents a command one or more of hearing instruments 102 to perform one or more actions (“YES branch of 706), assistance system 422 may perform one or more actions to execute the command. For example, assistance system 422 may generate computer-interpretable commands that instruct hearing instruments 102 to perform the one or more actions. Example commands may include increasing/decreasing the volume of one or more of hearing instruments 102, activating/deactivating/changing specific sound quality features of one or more of hearing instruments 102, activating/deactivating specific features combinations of parameters of hearing instruments 102, and so on. Thus, assistance system 422 may determine whether the voice input represents any command from user 104 to perform any action with respect to one or more of hearing instruments 102. Based on a determination that the voice input represents a command from user 104 to perform an action with respect to one or more of hearing instruments 102, assistance system 422 may transmit instructions to one or more of hearing instruments 102 that cause one or more of hearing instruments 102 to execute the command.

In the example of FIG. 7, if the voice input does not represent a command one or more of hearing instruments 102 to perform one or more actions (“NO” branch of 706), assistance system 422 may determine whether the voice input represents a request for help with respect to one or more of hearing instruments 102 (708). If the voice input represents a request for help with respect to one or more of hearing instruments 102 (“YES” branch of 708), help content system 414 (e.g., a help repository service) may generate a help response based on the intention of user 104. For example, help content system 414 may search a help content repository based on keywords or other information included in a hearing help question identified by NLP service 704 in the voice input or a subsequent voice input. In examples where the help response includes text and/or graphical data, help content system 414 may send instructions to local computing system 106 to show the help content. In some examples, help content system 414 may provide text to text-to-speech system 402. Text-to-speech system 402 may convert the text to speech audio data and provide the speech audio data to local computing system 106 for playback by local computing system 106 and/or one or more of hearing instruments 102. Thus, assistance system 422 may determine whether the voice input represents a request from user 104 for help with respect to one or more of hearing instruments 102. Based on a determination that the voice input represents a request from user 104 for help with respect to one or more of hearing instruments 102, help content system 414 may generate a help response that corresponds to the request from user 104 for help with respect to one or more of hearing instruments 102. In some examples, assistance system 422 outputs the help response for display by a display screen of a computing device. In some examples, assistance system 422 transmits, to one or more of hearing instruments 102, help response data that cause one or more of hearing instruments 102 to output sound representing the help response.

In some examples, such as the example of FIG. 7, help content system 414 generates the help response if the voice input does not represent a command and the voice input represents a help request. Thus, assistance system 422 may determine whether the voice input represents any command from user 104 to perform any action with respect to the one or more hearing instruments. Based on a determination that the voice input does not represent any command from user 104 to alter any aspect of the one or more hearing instruments, assistance system 422 may determine whether the voice input represents a request from user 104 for help with respect to one or more of hearing instruments 102. Based on a determination that the voice input represents a request from user 104 for help with respect to one or more of hearing instruments 102, help content system 414 may generate a help response that corresponds to the request from user 104 for help with respect to one or more of hearing instruments 102.

Furthermore, in the example of FIG. 7, if the voice input does not represent a request for help with respect to one or more of hearing instruments 102 (“NO” branch of 708), a control service 710 of virtual personal assistant 110 may coordinate services to provide an answer to a request from user 104. Control service 710 may be included in assistance system 422, or vice versa. In some examples, if the voice input from user 104 is not in a targeted language, control service 710 may use a language translation service 712 to translate the voice input into the targeted language. For example, if the targeted language is the English language, control service 710 may use language translation service 712 to translate the voice input into the English language.

In some examples, control service 710 may use real-time data service 420 to perform a real-time data process. Performance of the real-time data process may involve retrieval of information from online sources, such as webpages, application programming interfaces (APIs), and so on. Real-time does not necessarily mean derived at the exact time of the request but may refer to information that has not been captured in a general knowledge resource. For instance, the real-time information may be data beyond training information on which generative AI system 408 (e.g., LLM 409) was trained. Examples of general (static) information: What is the capital of Alaska? Who is the Governor of Delaware? When was the lithium ion battery invented? Examples of real-time information: What is the temperature today in Dana Point, California? Have there been whale sightings in Dana Point? What is the score of the local professional hockey game? How long is the wait time at my local urgent care clinics?

In some examples, assistance system 422 uses generative AI system 408 to determine whether the voice input represents a request for real-time information. For instance, in such examples, assistance system 422 may provide a first input to generative AI system 408. The first input requests that generative AI system 408 generate a first output that indicates whether the voice input is in a real-time information request class. Voice inputs in the real-time information request class may request information beyond training information on which the generative AI system was trained. Based on the first output indicating that the voice input is in the real-time information request class, assistance system 422 may send, via one or more communication links external to the computing system, a request for real-time data service 420 to provide real-time information specified by the voice input. For instance, local computing system 106 may send the request via a communication network, such as the Internet. Assistance system 422 may receive the real-time information from real-time data service 420. Assistance system 422 may provide a second input to generative AI system 408. The second input requests that generative AI system 408 generate a second output that expresses the real-time information in a conversational matter. For instance, the second input may request that generative AI system 408 express the real-time information as a personal assistant would provide the real-time information in a conversation between the personal assistant and user 104. Thus, in this example, the second input may include a prompt that directly specifies that generative AI system 408 is to generate an output based on the real-time information as a personal assistant would. In other examples, the second input may specify that generative AI system 408 generate the output as other types of people would. Assistance system 422 may transmit, to one or more of hearing instruments 102, response data that cause one or more of hearing instruments 102 to output sound representing the second output. For example, text-to-speech system 402 may generate audio data based on the second output and virtual personal assistant 110 may transmit the audio data to one or more of hearing instruments 102. In another example, virtual personal assistant 110 may transmit the second output in text form to one or more of hearing instruments 102, which may convert the text into audio data for playback.

In some examples, control service 710 may use generative AI system 408 to generate a response, e.g., as described elsewhere in this disclosure. For instance, generative AI system 408 may generate one or more queries based on the request and use one or more search engines 411 to execute the one or more queries. In this way, assistance system 422 may obtain audio data representing a voice input from the user. The voice input may be detected by the one or more microphones of one or more of hearing instruments 102. Assistance system 422 may provide a first input to generative AI system 408. The first input requests that generative AI system 408 generate a first output that indicates whether the voice input is in the real-time information request class. Based on the first output indicating that the voice input is not in the real-time information request class, assistance system 422 may provide a second input to generative AI system 408. The second input requests that generative AI system 408 generate a second output that expresses a response to the voice input. In some examples, the second input requests that generative AI system 408 generate the second output that expresses a response to the voice input as the personal assistant would provide the response in a conversation. Assistance system 422 may transmit, to one or more of hearing instruments 102, response data that cause one or more of hearing instruments 102 to output sound representing the second output. For example, text-to-speech system 402 may generate audio data based on the second output and virtual personal assistant 110 may transmit the audio data to one or more of hearing instruments 102. In another example, virtual personal assistant 110 may transmit the second output in text form to one or more of hearing instruments 102, which may convert the text into audio data for playback.

If a response generated by generative AI system 408 or real-time data service 420 is not in a language preferred by user 104, language translation service 712 may translate the response to the preferred language. Control service 710 may provide the response (which may be a translation of an initial response) to text-to-speech system 402. Text-to-speech system 402 may convert the response to speech audio data for playback by one or more of local computing system 106 or one or more of hearing instruments 102. Thus, in some examples, assistance system 422 may determine a source language. The source language is a language in which user 104 expressed the voice input. Based on the source language different from a target language of real-time data service, assistance system 422 may obtain a translated version of the voice input. The translated version of the voice input includes one or more words translated from the source language to the target language. Assistance system 422 may generate the request to real-time data service 420 based on the translated version of the voice input. Furthermore, in some examples, assistance system 422 may use language translation service 712 to translate the response to the source language.

In some examples, after the response to played back to user 104, virtual personal assistant 110 may receive additional input (e.g., requests) from one or more hearing instruments 102. In this way, user 104 may have a conversation with virtual personal assistant 110 without reinitiating the conversation (e.g., without double-tapping on one or more of hearing instruments 102 or opening a feature of local computing system 106 associated with virtual personal assistant 110). In some examples, virtual personal assistant 110 closes a conversation if virtual personal assistant 110 does not receive additional input without a predetermined time period, e.g., 10 seconds. In some examples, virtual personal assistant 110 closes a conversation if virtual personal assistant 110 detects a phrase that virtual personal assistant 110 recognizes as a phrase to close a conversation.

The following is a non-limiting list of clauses in accordance with one or more techniques of this disclosure.

Clause 1. A method comprising: receiving, by a computing system, audio data generated by one or more hearing instruments worn at or near one or more ears of a user; providing, by the computing system, a virtual personal assistant to the user, wherein the virtual personal assistant is configured to generate, based on the audio data, output to assist the user; and providing, by the computing system, the output to the one or more hearing instruments, wherein the one or more hearing instruments are configured to generate auditory stimuli based on the output.

Clause 2. The method of clause 1, wherein: providing the virtual personal assistant comprises applying, by the computing system, a Large Language Model (LLM) to generate a response, and the output is based on the response.

Clause 3. The method of clause 2, wherein: providing the virtual personal assistant comprises generating, by the computing system, a prompt based on the audio data, and applying the LLM comprises applying, by the computing system, the LLM to the prompt to generate the response.

Clause 4. The method of any of clauses 1-3, wherein the audio data is first audio data and the one or more hearing instruments are configured to generate the first audio data by applying signal processing to second audio data generated by microphones of the one or more hearing instruments.

Clause 5. The method of any of clauses 1-4, wherein the virtual personal assistant is configured to learn a routine of the user based at least in part on the audio data and generate the output based on the routine of the user.

Clause 6. The method of clause 5, wherein the output based on the routine of the user includes a reminder to perform an activity.

Clause 7. The method of any of clauses 1-6, wherein the virtual personal assistant is configured to determine, based on the audio data, whether an event has occurred and to generate the output indicating whether the event has occurred.

Clause 8. The method of any of clauses 1-7, wherein the event is the user taking medication.

Clause 9. The method of any of clauses 1-8, wherein the virtual personal assistant is configured to access a calendar and the output is based on events in the calendar.

Clause 10. The method of any of clauses 1-9, wherein the audio data represent a voice of a person with whom the user is interacting, and the output generated by the virtual personal assistant includes information about the person.

Clause 11. The method of clause 10, wherein the information about the person includes information about interactions between the person and the user.

Clause 12. The method of any of clauses 10-11, wherein the virtual personal assistant is configured to learn the information about the person based on the audio data received from the one or more hearing instruments.

Clause 13. The method of any of clauses 1-12, wherein the output generated by the virtual personal assistant includes a recommended or automatic adjustment to one or more aspects of the one or more hearing instruments.

Clause 14. The method of clause 13, wherein the audio data includes a request from the user to improve sound quality of the one or more hearing instruments.

Clause 15. The method of any of clauses 1-14, wherein the virtual personal assistant is configured to receive health data for the user.

Clause 16. The method of any of clauses 1-15, wherein providing the virtual personal assistant comprises extracting, by the computing system, semantic content of speech represented by the audio data.

Clause 17. A computing system comprising one or more memories; and one or more processors configured to: receive audio data generated by one or more hearing instruments worn at or near one or more ears of a user; provide a virtual personal assistant to the user, wherein the virtual personal assistant is configured to generate, based on the audio data, output to assist the user; and provide the output to the one or more hearing instruments, wherein the one or more hearing instruments are configured to generate auditory stimuli based on the output.

Clause 18. The computing system of clause 17, wherein the one or more processors are configured to perform the methods of any of clauses 2-16.

Clause 19. One or more non-transitory computer-readable media having instructions stored thereon that, when executed by one or more processors of a computing system, cause the one or more processors to: receive audio data generated by one or more hearing instruments worn at or near one or more ears of a user; provide a virtual personal assistant to the user, wherein the virtual personal assistant is configured to generate, based on the audio data, output to assist the user; and provide the output to the one or more hearing instruments, wherein the one or more hearing instruments are configured to generate auditory stimuli based on the output.

Clause 20. The one or more non-transitory computer-readable media of clause 19, wherein the one or more processors are configured to perform the methods of any of clauses 2-16.

Clause 21. A method comprising: generating, by a hearing instrument worn at or near one or more ears of a user, audio data; sending, by the hearing instrument, the audio data to a computing system that provides a virtual personal assistant to the user, wherein the virtual personal assistant is configured to generate, based on the audio data, output to assist the user; and receiving, by the hearing instrument, the output; and generating, by the hearing instrument, auditory stimuli based on the output.

Clause 22. The method of clause 21, wherein the hearing instrument is configured to use with the methods of any of clauses 2-16.

Clause 23. A hearing instrument configured to perform the methods of any of clauses 21-22.

Clause 24. A non-transitory computer-readable medium having instructions stored thereon that, when executed, cause one or more processors of a hearing instrument to perform the methods of any of clauses 21-22.

Clause 25. A computing system comprising: one or more processors; and one or more memories comprising processor-executable instructions that, when executed by the one or more processors, configure the one or more processors to: obtain audio data representing a voice input from a user of one or more hearing instruments, wherein the voice input is detected by one or more microphones of the one or more hearing instruments; provide a first input to a generative artificial intelligence (AI) system, wherein the first input requests that the generative AI system generate a first output that indicates whether the voice input is in a real-time information request class, wherein voice inputs in the real-time information request class request information beyond training information on which the generative AI system was trained; based on the first output indicating that the voice input is in the real-time information request class, send, via one or more communication links external to the computing system, a request for a real-time data service to provide real-time information specified by the voice input; receive the real-time information from the real-time data service; provide a second input to the generative AI system, wherein the second input requests that the generative AI system generate a second output that expresses the real-time information as a personal assistant would provide the real-time information in a conversation between the personal assistant and the user; and transmit, to the one or more hearing instruments, response data that cause the one or more hearing instruments to output sound representing the second output.

Clause 26. The computing system of clause 25, wherein the one or more processors are further configured to: determine whether the voice input represents any command from the user to perform any action with respect to the one or more hearing instruments; and based on a determination that the voice input represents a command from the user to perform an action with respect to the one or more hearing instruments, transmit instructions to the one or more hearing instruments that cause the one or more hearing instruments to execute the command.

Clause 27. The computing system of clause 26, wherein the command is to change an output volume of the one or more hearing instruments or to change a sound quality feature of the one or more hearing instruments.

Clause 28. The computing system of clause 25, wherein the one or more processors are further configured to: determine whether the voice input represents a request from the user for help with respect to the one or more hearing instruments; and based on a determination that the voice input represents a request from the user for help with respect to the one or more hearing instruments, generate a help response that corresponds to the request from the user for help with respect to the one or more hearing instruments.

Clause 29. The computing system of clause 28, wherein one or more processors are further configured to at least one of: output the help response for display by a display screen of a computing device, or transmit, to the one or more hearing instruments, help response data that cause the one or more hearing instruments to output sound representing the help response.

Clause 30. The computing system of clause 25, wherein the one or more processors are further configured to: determine whether the voice input represents any command from the user to perform any action with respect to the one or more hearing instruments; based on a determination that the voice input does not represent any command from the user to alter any aspect of the one or more hearing instruments, determine whether the voice input represents a request from the user for help with respect to the one or more hearing instruments; and based on a determination that the voice input represents a request from the user for help with respect to the one or more hearing instruments, generate a help response that corresponds to the request from the user for help with respect to the one or more hearing instruments.

Clause 31. The computing system of clause 25, wherein the audio data is first audio data, the voice input is a first voice input, the request for real-time information is a first request for first real-time information, the response data is first response data, and the one or more processors are further configured to: obtain second audio data representing a second voice input from the user, wherein the second voice input is detected by the one or more microphones of the one or more hearing instruments; provide a third input to the generative AI system, wherein the third input requests that the generative AI system generate a third output that indicates whether the second voice input is in the real-time information request class; and based on the third output indicating that the second voice input is not in the real-time information request class, provide a fourth input to the generative AI system, wherein the fourth input requests that the generative AI system generate a fourth output that expresses a response to the second voice input as the personal assistant would provide the response in the conversation; and transmit, to the one or more hearing instruments, second response data that cause the one or more hearing instruments to output sound representing the fourth output.

Clause 32. The computing system of clause 25, wherein the one or more processors are further configured to: determine a source language, wherein the source language is a language in which the user expressed the voice input; and based on the source language different from a target language of the real-time data service: obtain a translated version of the voice input, wherein the translated version of the voice input includes one or more words translated from the source language to the target language; and generate the request based on the translated version of the voice input.

Clause 33. The computing system of clause 25, wherein the computing system includes a local computing device that includes the one or more processors.

Clause 34. The computing system of clause 33, wherein: the one or more processors are configured to transmit the first input to a remote computing system that hosts the generative AI system, and the one or more processors are configured to transmit the second input to the remote computing system.

Clause 35. A computer-implemented method comprising: obtaining, by one or more processors of a computing system, audio data representing voice input from a user of one or more hearing instruments, wherein the voice input is detected by one or more microphones of the one or more hearing instruments; providing, by the one or more processors, a first input to a generative artificial intelligence (AI) system, wherein the first input requests that the generative AI system generate a first output that indicates whether the voice input is in a real-time information request class, wherein voice inputs in the real-time information request class request information beyond training information on which the generative AI system was trained; based on the first output indicating that the voice input is in the real-time information request class, sending, by the one or more processors, via one or more communication links external to the computing system, a request for a real-time data service to provide real-time information specified by the voice input; receiving, by the one or more processors, the real-time information from the real-time data service; providing, by the one or more processors, a second input to the generative AI system, wherein the second input requests that the generative AI system generate a second output that expresses the real-time information as a personal assistant would provide the real-time information in a conversation between the personal assistant and the user; and transmitting, by the one or more processors, to the one or more hearing instruments, response data that cause the one or more hearing instruments to output sound representing the second output.

Clause 36. The computer-implemented method of clause 35, wherein the method further comprises: determining, by the one or more processors, whether the voice input represents any command from the user to perform any action with respect to the one or more hearing instruments; and based on a determination that the voice input represents a command from the user to perform an action with respect to the one or more hearing instruments, transmitting, by the one or more processors, instructions to the one or more hearing instruments that cause the one or more hearing instruments to execute the command.

Clause 37. The computer-implemented method of clause 36, wherein the command is to change an output volume of the one or more hearing instruments or to change a sound quality feature of the one or more hearing instruments.

Clause 38. The computer-implemented method of clause 35, further comprising: determining, by the one or more processors, whether the voice input represents a request from the user for help with respect to the one or more hearing instruments; and based on a determination that the voice input represents a request from the user for help with respect to the one or more hearing instruments, generating, by the one or more processors, a help response that corresponds to the request from the user for help with respect to the one or more hearing instruments.

Clause 39. The computer-implemented method of clause 38, further comprising at least one of: outputting, by the one or more processors, the help response for display by a display screen of a computing device, or transmitting, by the one or more processors, to the one or more hearing instruments, help response data that cause the one or more hearing instruments to output sound representing the help response.

Clause 40. The computer-implemented method of clause 35, further comprising: determining, by the one or more processors, whether the voice input represents any command from the user to perform any action with respect to the one or more hearing instruments; based on a determination that the voice input does not represent any command from the user to alter any aspect of the one or more hearing instruments, determining, by the one or more processors, whether the voice input represents a request from the user for help with respect to the one or more hearing instruments; and based on a determination that the voice input represents a request from the user for help with respect to the one or more hearing instruments, generating, by the one or more processors, a help response that corresponds to the request from the user for help with respect to the one or more hearing instruments.

Clause 41. The computer-implemented method of clause 35, wherein the audio data is first audio data, the voice input is a first voice input, the request for real-time information is a first request for first real-time information, the response data is first response data, and the method further comprises: obtaining, by the one or more processors, second audio data representing second voice input from the user, wherein the second voice input is detected by the one or more microphones of the one or more hearing instruments; providing, by the one or more processors, a third input to the generative AI system, wherein the third input requests that the generative AI system generate a third output that indicates whether the second voice input is in the real-time information request class; and based on the third output indicating that the second voice input is not in the real-time information request class, providing, by the one or more processors, a fourth input to the generative AI system, wherein the fourth input requests that the generative AI system generate a fourth output that expresses a response to the second voice input as the personal assistant would provide the response in the conversation; and transmitting, by the one or more processors, to the one or more hearing instruments, second response data that cause the one or more hearing instruments to output sound representing the fourth output.

Clause 42. The computer-implemented method of clause 35, further comprising: determining, by the one or more processors, a source language, wherein the source language is a language in which the user expressed the voice input; and based on the source language different from a target language of the real-time data service: obtaining, by the one or more processors, a translated version of the voice input, wherein the translated version of the voice input includes one or more words translated from the source language to the target language; and generating, by the one or more processors, the request based on the translated version of the voice input.

Clause 43. The computer-implemented method of clause 35, wherein a local computing device includes the one or more processors.

Clause 44. The computer-implemented method of clause 43, wherein: providing the first input to the generative AI system comprises transmitting, by the one or more processors, the first input to a remote computing system that hosts the generative AI system. and providing the second input to the generative AI system comprises transmitting, by the one or more processors, the second input to the remote computing system.

Clause 45. A non-transitory computer-readable medium having instructions stored thereon that, when executed, cause one or more processors of a hearing instrument to perform the methods of any of clauses 35-44.

It is to be recognized that depending on the example, certain acts or events of any of the techniques described herein can be performed in a different sequence, may be added, merged, or left out altogether (e.g., not all described acts or events are necessary for the practice of the techniques). Moreover, in certain examples, acts or events may be performed concurrently, e.g., through multi-threaded processing, interrupt processing, or multiple processors, rather than sequentially.

In one or more examples, the functions described may be implemented in hardware, software, firmware, or any combination thereof. If implemented in software, the functions may be stored on or transmitted over, as one or more instructions or code, a computer-readable medium and executed by a hardware-based processing unit. Computer-readable media may include computer-readable storage media, which corresponds to a tangible medium such as data storage media, or communication media including any medium that facilitates transfer of a computer program from one place to another, e.g., according to a communication protocol. In this manner, computer-readable media generally may correspond to (1) tangible computer-readable storage media which is non-transitory or (2) a communication medium such as a signal or carrier wave. Data storage media may be any available media that can be accessed by one or more computers or one or more processing circuits to retrieve instructions, code and/or data structures for implementation of the techniques described in this disclosure. A computer program product may include a computer-readable medium.

By way of example, and not limitation, such computer-readable storage media can comprise RAM, ROM, EEPROM, CD-ROM or other optical disk storage, magnetic disk storage, or other magnetic storage devices, flash memory, cache memory, or any other medium that can be used to store desired program code in the form of instructions or data structures and that can be accessed by a computer. Also, any connection may be considered a computer-readable medium. For example, if instructions are transmitted from a website, server, or other remote source using a coaxial cable, fiber optic cable, twisted pair, digital subscriber line (DSL), or wireless technologies such as infrared, radio, and microwave, then the coaxial cable, fiber optic cable, twisted pair, DSL, or wireless technologies such as infrared, radio, and microwave are included in the definition of medium. It should be understood, however, that computer-readable storage media and data storage media do not include connections, carrier waves, signals, or other transient media, but are instead directed to non-transitory, tangible storage media. Combinations of the above should also be included within the scope of computer-readable media.

Functionality described in this disclosure may be performed by fixed function and/or programmable processing circuitry. For instance, instructions may be executed by fixed function and/or programmable processing circuitry. Such processing circuitry may include one or more processors, such as one or more digital signal processors (DSPs), general purpose microprocessors, application specific integrated circuits (ASICs), field programmable logic arrays (FPGAs), or other equivalent integrated or discrete logic circuitry. Accordingly, the term “processor,” as used herein may refer to any of the foregoing structure or any other structure suitable for implementation of the techniques described herein. In addition, in some aspects, the functionality described herein may be provided within dedicated hardware and/or software modules. Also, the techniques could be fully implemented in one or more circuits or logic elements. Processing circuits may be coupled to other components in various ways. For example, a processing circuit may be coupled to other components via an internal device interconnect, a wired or wireless network connection, or another communication medium.

Various components, modules, or units are described in this disclosure to emphasize functional aspects of devices configured to perform the disclosed techniques, but do not necessarily require realization by different hardware units. Rather, as described above, various units may be combined in a hardware unit or provided by a collection of interoperative hardware units, including one or more processors as described above, in conjunction with suitable software and/or firmware.

Various examples have been described. These and other examples are within the scope of the following claims.

What is claimed is:

1. A method comprising:
receiving, by a computing system, audio data generated by one or more hearing instruments worn at or near one or more ears of a user;
providing, by the computing system, a virtual personal assistant to the user, wherein the virtual personal assistant is configured to generate, based on the audio data, output to assist the user, wherein providing the virtual personal assistant comprises applying, by the computing system, a Large Language Model (LLM) to generate a response, and the output is based on the response; and
providing, by the computing system, the output to the one or more hearing instruments, wherein the one or more hearing instruments are configured to generate auditory stimuli based on the output.

2. The method of claim 1, wherein:
providing the virtual personal assistant comprises generating, by the computing system, a prompt based on the audio data, and
applying the LLM comprises applying, by the computing system, the LLM to the prompt to generate the response.

3. The method of claim 1, wherein the audio data is first audio data and the one or more hearing instruments are configured to generate the first audio data by applying signal processing to second audio data generated by microphones of the one or more hearing instruments.

4. The method of claim 1, wherein the virtual personal assistant is configured to learn a routine of the user based at least in part on the audio data and generate the output based on the routine of the user.

5. The method of claim 4, wherein the output based on the routine of the user includes a reminder to perform an activity.

6. The method of claim 1, wherein the virtual personal assistant is configured to determine, based on the audio data, whether an event has occurred and to generate the output indicating whether the event has occurred.

7. The method of claim 6, wherein the event is the user taking medication.

8. The method of claim 1, wherein the virtual personal assistant is configured to access a calendar and the output is based on events in the calendar.

9. The method of claim 1, wherein the audio data represent a voice of a person with whom the user is interacting, and the output generated by the virtual personal assistant includes information about the person.

10. The method of claim 9, wherein the information about the person includes information about interactions between the person and the user.

11. The method of claim 9, wherein the virtual personal assistant is configured to learn the information about the person based on the audio data received from the one or more hearing instruments.

12. The method of claim 1, wherein the output generated by the virtual personal assistant includes a recommended or automatic adjustment to one or more aspects of the one or more hearing instruments.

13. The method of claim 12, wherein the audio data includes a request from the user to improve sound quality of the one or more hearing instruments.

14. The method of claim 1, wherein the virtual personal assistant is configured to receive health data for the user.

15. The method of claim 1, wherein providing the virtual personal assistant comprises extracting, by the computing system, semantic content of speech represented by the audio data.

16. A computing system comprising:
one or more memories; and
one or more processors configured to:
receive audio data generated by one or more hearing instruments worn at or near one or more ears of a user;
provide a virtual personal assistant to the user, wherein the virtual personal assistant is configured to generate, based on the audio data, output to assist the user, wherein as part of providing the virtual personal assistant, the one or more processors apply a Large Language Model (LLM) to generate a response, and the output is based on the response; and
provide the output to the one or more hearing instruments, wherein the one or more hearing instruments are configured to generate auditory stimuli based on the output.

17. The computing system of claim 16, wherein the audio data represent a voice of a person with whom the user is interacting, and the output generated by the virtual personal assistant includes information about the person.

18. The computing system of claim 16, wherein the output generated by the virtual personal assistant includes a recommended or automatic adjustment to one or more aspects of the one or more hearing instruments.

19. The computing system of claim 17, wherein the audio data includes a request from the user to improve sound quality of the one or more hearing instruments.

20. One or more non-transitory computer-readable media having instructions stored thereon that, when executed by one or more processors of a computing system, cause the one or more processors to:
receive audio data generated by one or more hearing instruments worn at or near one or more ears of a user;
provide a virtual personal assistant to the user, wherein the virtual personal assistant is configured to generate, based on the audio data, output to assist the user, wherein providing the virtual personal assistant comprises applying a Large Language Model (LLM) to generate a response, and the output is based on the response; and
provide the output to the one or more hearing instruments, wherein the one or more hearing instruments are configured to generate auditory stimuli based on the output.
